(19) United States
(12) Patent Application Publication
(10) Pub. No.: US 2023/0211052 A1
(43) Pub. Date: Jul. 6, 2023

(54) COMPOUNDED ACTIVE PHARMACEUTICAL AGENTS IN THERMOPLASTIC POLYMER COMPOSITIONS AND METHODS OF MANUFACTURE

(71) Applicant: TELEFLEX MEDICAL INCORPORATED, Morrisville, NC (US)

(72) Inventors: Nisha GUPTA, Reading, PA (US); Kevin SECHRIST, Reading, PA (US); Eric MARCHESE, Reading, PA (US)

(21) Appl. No.: 18/090,421

(22) Filed: Dec. 28, 2022

Related U.S. Application Data
(60) Provisional application No. 63/295,132, filed on Dec. 30, 2021.

(51) Int. Cl. A61L 29/16; A01N 47/44; A01N 25/10; A01N 25/12; A61L 29/06; B29B 7/48; B29B 7/82

(52) U.S. Cl. CPC A61L 29/16; A01N 47/44; A01N 25/10; A01N 25/12; A61L 29/06; B29B 7/48; B29B 7/82; A61L 2300/404; A61L 2300/42; A61L 2300/41; A61L 2300/206; B29K 2105/0035

(57) ABSTRACT

In a method of integrating an active pharmaceutical ingredient (API) with a thermoplastic polymer, the thermoplastic polymer and API are into a first feed port of a multi-screw extruder or the thermoplastic polymer is fed into the first feed port of a multi-screw extruder, the thermoplastic polymer is conveyed along the heated multi-screw extruder while heating the thermoplastic polymer to a melt temperature of 160° C.-280° C. prior to the thermoplastic polymer being conveyed past a second feed port and the API is fed into the second feeding port in the heated screw extruder to mix with the melted thermoplastic polymer to generate a compounded mixture containing 85-100% of the starting API content. The compounded mixture is extruded from an outlet of the heated screw extruder and cooled via a cooling device such that the compounded mixture contains 85-100% of the starting API content.

### CROSS REFERENCE TO RELATED APPLICATIONS

This application claims the benefit of U.S. Application no. priority to Chinese Patent Application No. 63/295,132, filed Dec. 30, 2021, the disclosure of which is incorporated by reference in its entirety.

### FIELD OF THE INVENTION

The present invention generally relates to compounded polyurethane compositions for medical devices having antimicrobial, antithrombogenic, and/or anti-inflammatory properties. More particularly, the present invention pertains to melt processable polyurethane compositions for medical devices having antimicrobial, antithrombogenic, and/or anti-inflammatory properties and method of production thereof.

### BACKGROUND OF THE INVENTION

Medical devices are commonly used to facilitate care and treatment of patients undergoing surgical procedures. Examples of such devices include catheters, grafts, stents, sutures, and the like. Unfortunately, organisms such as bacteria and fungi may infiltrate and/or form biofilms on these medical devices which may be difficult to treat. Such contamination may lead to infections and cause discomfort or illness.

It is generally known that in various medical procedures, the use of medical devices having antimicrobial properties may reduce the incidence of infection in the patient. Typically, the antimicrobial agent is applied as a coating on the conventional medical device or the antimicrobial agent is infused into the conventional medical device by soaking the device in a solution of the antimicrobial agent. In these and other conventional methods of introducing the antimicrobial agent to the medical device, this extra step of coating or soaking takes time and increases costs.

In addition to the added step and increased production time, soaking and coating may not achieve relatively high concentrations of antibiotic in the base material of the medical device. For relatively short procedures having a duration of a few hours, this relatively low antibiotic concentration may be sufficient. However, for longer procedures lasting several days, the antibiotic present in conventional devices may be insufficient. As such, these conventional devices must be replaced frequently as the antibiotic falls below effective levels.

Accordingly, it is desirable to provide an antimicrobial medical device and/or method of introducing an antimicrobial agent to a medical device that is capable of overcoming the disadvantages described herein at least to some extent.

### SUMMARY OF THE INVENTION

The foregoing needs are met, to a great extent, by the present invention, wherein in one respect a polymer is compounded with an active pharmaceutical ingredient (API) for a medical device and method of compounding the polymer and API is provided.

An embodiment of the present invention pertains to a method of integrating an active pharmaceutical ingredient (API) with a thermoplastic polymer. The method includes: feeding the thermoplastic polymer and API into a first feed port of a multi-screw extruder; or feeding the thermoplastic polymer into a first feed port of a twin-screw extruder; conveying the thermoplastic polymer along the heated multi-screw extruder; heating the thermoplastic polymer to a melt temperature of 160° C.-280° C. prior to the thermoplastic polymer being conveyed past a second feed port; the second feed port is feeding the API into the heated screw extruder to mix with the melted thermoplastic polymer to generate a compounded mixture containing 85-100% of the starting API content; extruding the compounded mixture from an outlet of the heated screw extruder; and passing the extruded compounded mixture through a cooling device to cool the extruded compounded mixture such that the compounded mixture contains 85-100% of the starting API content.

Another embodiment of the present invention relates to a medical device. The medical device includes a thermoplastic polymer integrated with an active pharmaceutical ingredient (API). A method of integrating the API with the thermoplastic polymer includes: feeding the thermoplastic polymer and API into a first feed port of a twin-screw extruder; or feeding the thermoplastic polymer into a first feed port of a twin-screw extruder, conveying the thermoplastic polymer along the heated multi-screw extruder, heating the thermoplastic polymer to a melt temperature of 160° C.-280° C. prior to the thermoplastic polymer being conveyed past a second feed port; the second feed port is feeding the API into the heated multi-screw extruder to mix with the melted thermoplastic polymer to generate a compounded mixture containing 85-100% of the starting API content; extruding the compounded mixture from an outlet of the heated screw extruder; and passing the extruded compounded mixture through a cooling device to cool the extruded compounded mixture such that the compounded mixture contains 85-100% of the starting API content.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a diagram of a system for compounding a thermo-polymer with an active pharmaceutical ingredient (API).

FIG. 2 is a chart of API content per resin configuration.

FIG. 3 is a chart of API elution over time.

FIG. 4 is a chart of API content per resin configuration.

FIG. 5 is a chart of API content over time.

FIG. 6 is a chart of API content over time.

FIG. 7 is a chart of API content over time.

FIG. 8 is a high performance liquid chromatograph showing an analysis at a wavelength of 280 nm of a chlorhexidine diacetate (CHA) heated to a temperature of 210° C. for 10 minutes.

FIG. 9 is a high performance liquid chromatograph showing an analysis at a wavelength of 280 nm of unheated CHA.

FIG. 10 is a high performance liquid chromatograph showing an analysis at a wavelength of 280 nm of a chlorhexidine dihydrochloride (CHD) heated to a temperature of 210° C. for 10 minutes.

FIG. 11 is a high performance liquid chromatograph showing an analysis at a wavelength of 280 nm of unheated CHD.

FIG. 12 is a simplified view of an extruder and air-cooling device according to an embodiment of the invention.

FIG. 13 is a plot of actual API percentages is different polymer formulations after compounding and water cooling.

FIG. 14 is a plot of API percentages after compounding and air-cooling.

FIG. 15 is a plot of API percentages after compounding and air-cooling.

### DETAILED DESCRIPTION

Embodiments of the invention provide a system and device for compounding an active pharmaceutical ingredient (API) into a polymer. Examples of APIs include active antimicrobial agents, antithrombogenic agents, anti-inflammatory agents, and the like. Particular examples of suitable antimicrobial agents include biguanides such as chlorhexidine and Alexidine. Examples of suitable polymers include thermoplastic polymers having a melt temperature of 160° C.-280° C.

When compounding an API in an extruder, such as a twin or multi screw extruder, the thermoplastic polymer is heated to the melt temperature of the polymer. Once melted, the polymer remains in the melted state until the temperature falls to the solidification temperature. Depending on the polymer, there may be several degrees Celsius separating these states. As described herein, the API is introduced downstream from the polymer inlet. It is an advantage that this action introduces the API to the melted polymer at a portion of the screw extruder that is not actively heating the polymer to the melt temperature and may be cooler than the upstream portion of the extruder. In addition, by subjecting the API to the elevated temperature of the melted polymer for a shorter duration, the API may experience less thermal degradation.

The compounded polymer and API is quickly cooled after thorough mixing and extrusion. However, it has surprisingly been found that some methods of cooling in water can cause a significant loss of API from the compounded polymer. For the purpose of this disclosure, a significant loss of API is a loss of API that is 15% or greater. In addition to the added cost of the lost API, increasing the initial amount of API added to the polymer may have adverse effects such as clouding, crystallization of the API, and the like. For example, if water is used for cooling, the exposure time has to be minimized to prevent significant loss of API. Alternatively, it has been advantageously found that the use of a sufficient amount of air-cooling has the same cooling performance while retaining the API in the compounded polymer.

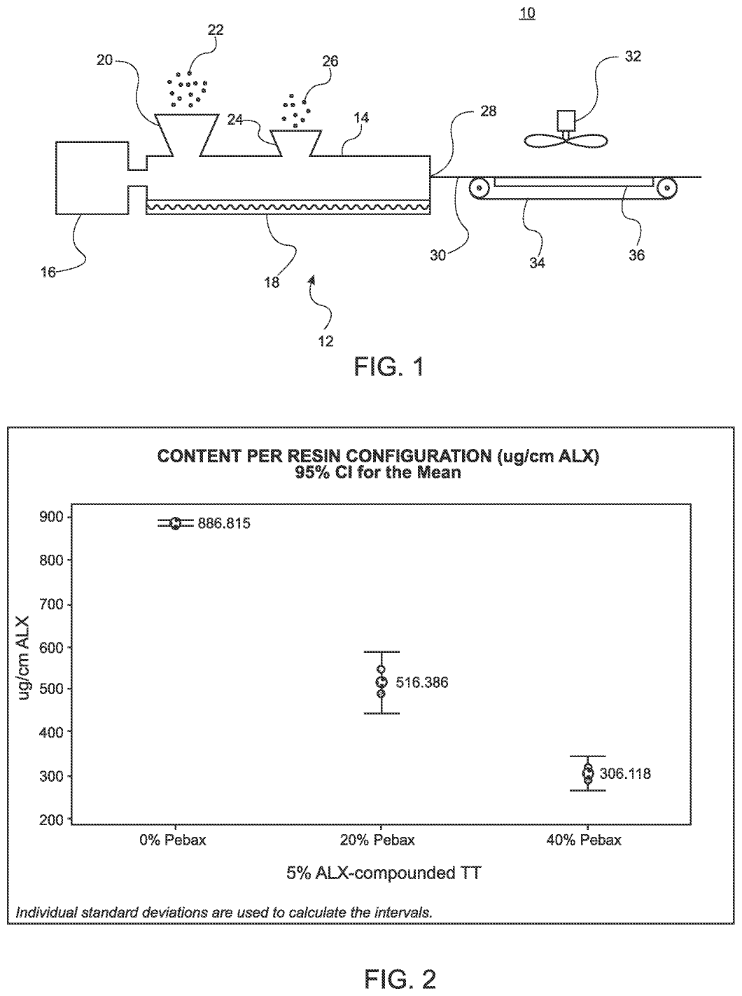

FIG. 1 is a diagram of a system 10 for compounding a thermo-polymer with an active pharmaceutical ingredient (API). As shown in FIG. 1, the system 10 includes an extruder 12 with a body 14, a motor 16 to turn internal screws (not shown), and a heater 18. The body 14 includes a first port 20 for introducing a polymer 22. The body 14 includes a second port 24 for introducing an API 26. As shown, the API 26 is introduced downstream from the first port 20 and the heater 18. In some examples, the second port 24 is disposed at least halfway along a length of the body 14.

The compounding mixture of the polymer 22 and API 26 is urged toward an outlet 28 as it is mixed. Once mixed and extruded through the outlet 28, a compounded mixture 30 is cooled via an air-cooling device 32. In some examples, the air-cooling device 32 includes one or more air rings. In other examples, the air-cooling device 32 includes one or more fans. Optionally, the system 10 may include a conveyer belt 34 to convey the compounded mixture 30 from the outlet 28. A chilled platen 36 may be configured to cool the conveyer belt 34 and, thereby, facilitate cooling of the compounded mixture 30. In various examples, the conveyer belt 34 may include a thermally conductive material such as, for example, stainless steel. The chilled platen 36 may include tubing for a flow of chilled water or refrigerant or the chilled platen 36 may include a piezoelectric chiller to provide cooling.

In some examples, the compounded mixture 30 is extruded into a medical device, such as medical tubing, a stent, a catheter or the like. In other examples, the compounded mixture 30 is processed into pellets for further processing into a medical device.

More particularly, the present invention relates to medical device composed of materials that allow the device to impart long term antimicrobial, antithrombogenic and anti-inflammatory effects due to the API releasing from the device for the period the device resides in body for a clinical indication; the said medical device is composed by using a method which integrates the antimicrobial biguanide agents (chlorhexidine, alexidine, octinedine) and a hydrophilic material such as Polyether polyurethane with PEG or a polyether block amide material in to the bulk device polymeric matrix enhancing the release of the antimicrobial agent from the device.

The polymers are aromatic polyurethanes (Tecothane, Isoplast), and aliphatic polyurethanes (e.g. Tecoflex, Carbothane, Quadrathane), the antimicrobials are chlorhexidine, alexidine, octinedine, and the hydrophilic polymers (e.g. PEBAX—Polyether Block Amide material, Tecophillic—Polyether polyurethane with PEG as its poly-ol). A device consisting of a polymer matrix composed of one of the following combinations allowing controlled release of the antimicrobial agent over a long period of time. Some examples of suitable compounded polyurethane API mixtures include: Aliphatic polyurethane+Antimicrobial agent+Polyether block Amide; Aromatic polyurethane+Antimicrobial agent+Polyether block Amide; Aliphatic polycarbonate polyurethane+Antimicrobial agent+Polyether block Amide; Aromatic polycarbonate polyurethane+Antimicrobial agent+Polyether block Amide; and Aromatic Polycarbonate silicone polyurethane+Antimicrobial agent+Polyether block Amide

A suitable medical device for use with the compounded mixture of the present invention may be adapted for contact with a vessel or cavity in the body. Examples of suitable polymers may be aromatic or aliphatic polyurethanes with bulk distributed antimicrobial compound with a melt temperature above 200° C., the amount of antimicrobial agent is 0.5-15.0 wt/wt % and a bulk distributed hydrophilic polymer which results in a moisture uptake by the device at 5-35 wt/wt %, which results in both anti-thrombogenic and anti-microbial effects from the device. The antimicrobial agents include biguanide class of antimicrobials with a melt temperature above 200° C., e.g. CHX-DH (Chlorhexidine dihydrochloride) and ALX-DH (Alexidine dihydrochloride). The antimicrobial agents preferably include biguanide class of antimicrobials which remains stable and do not degrade at temperature below 200° C.

To control the elution rate of the compounded API, the bulk distributed hydrophilic polymer preferably has at least have moisture uptake of 15-50% resulting in 5-35% moisture uptake from the device. In this manner, the medical device facilitates a release of API at least 1% of the total loading of the API. In preferred examples, the medical device is constructed using a compounding process that maintains temperature below 200° C.

As described herein, the compounding process includes chilling agent or process, that excludes water. As described herein, use of water to chill the compounded mixture results in a loss of about 50% of the API from the compounded mixture. As such, air-cooling is the preferred agent or process to cool the extrudate into the medical device or to a temperature that is conducive to cutting into pellets.

### Example 1: Tecothane+ALX+PBAX (0%, 20% and 40%)—Formulation Composition, Content, Elution, Antimicrobial Efficacy

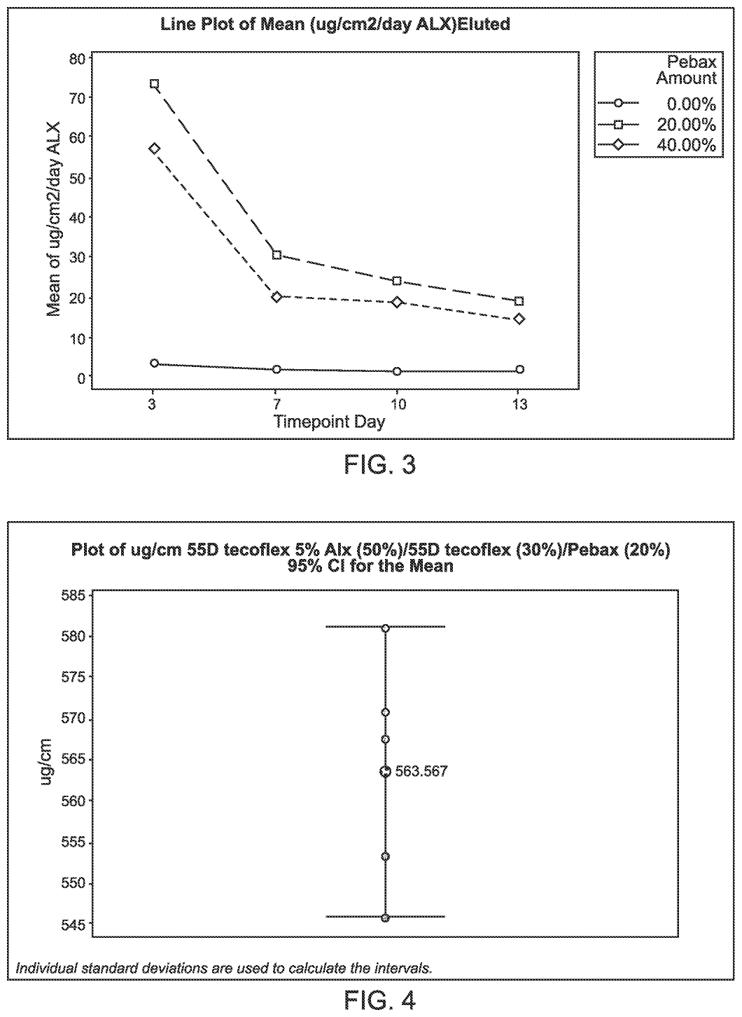

Tecothane polyurethane material was compounded with 5% Alexidine followed by extruding to form 7french 3-lumen catheters, and tested for Content, Elution, and Efficacy. The alexidine content results were 887 μg/cm. When these catheters were tested for antimicrobial efficacy, the performance was poor because the elution rate was low. To enhance Alexidine elution, hydrophilic material, PEBAX, was added at 20% and 40% ratio during the compounding process. FIG. 2 shows the content of each of the blends. FIG. 3 shows the results of the elution testing. Adding the 20% and 40% PEBAX had an effect that enhanced the elution rate of the Alexidine. Table 1 shows the results of the Efficacy testing against C. albicans, E. faecalis, and K. pneumoniae, 20% and 40% had greater than 4 log reduction on day 14 challenge.

TABLE 1
Antimicrobial Efficacy of the external surface of
extrusions composed of Tecothane + ALX + PBAX (20% and 40%)
LR 2019-014: External Efficacy Experiment 150

| | | 5% Alexidine + 20% PEBAX | | 5% Alexidine + 40% PEBAX | |
|---|---|---|---|---|---|
| | | | Log₁₀ | | Log10 |
| | | | Reduction | | Reduction |
| | Day 14 | CFU/Segment | Control | CFU/Segment | Control |
| Candida | Replicate 1 | 0.00E+00 | 5.4 | 0.00E+00 | 5.4 |
| albicans | Replicate 2 | 0.00E+00 | | 0.00E+00 | |
| ATCC 10231 | Replicate 3 | 0.00E+00 | | 0.00E+00 | |
| Enterococcus | Replicate 1 | 0.00E+00 | 5.8 | 6.13E+01 | 4.4 |
| faecalis | Replicate 2 | 0.00E+00 | | 0.00E+00 | |
| ATCC 51299 | Replicate 3 | 0.00E+00 | | 1.83E+02 | |
| Klebsiella | Replicate 1 | 0.00E+00 | 4.3 | 1.33E+00 | 4.3 |
| pneumoniae | Replicate 2 | 0.00E+00 | | 0.00E+00 | |
| ATCC 10031 | Replicate 3 | 0.00E+00 | | 0.00E+00 | |

### Example 2: Tecoflex+ALX+PBAX (0%, 20%)—Formulation Composition, Content, Elution, Antimicrobial Efficacy

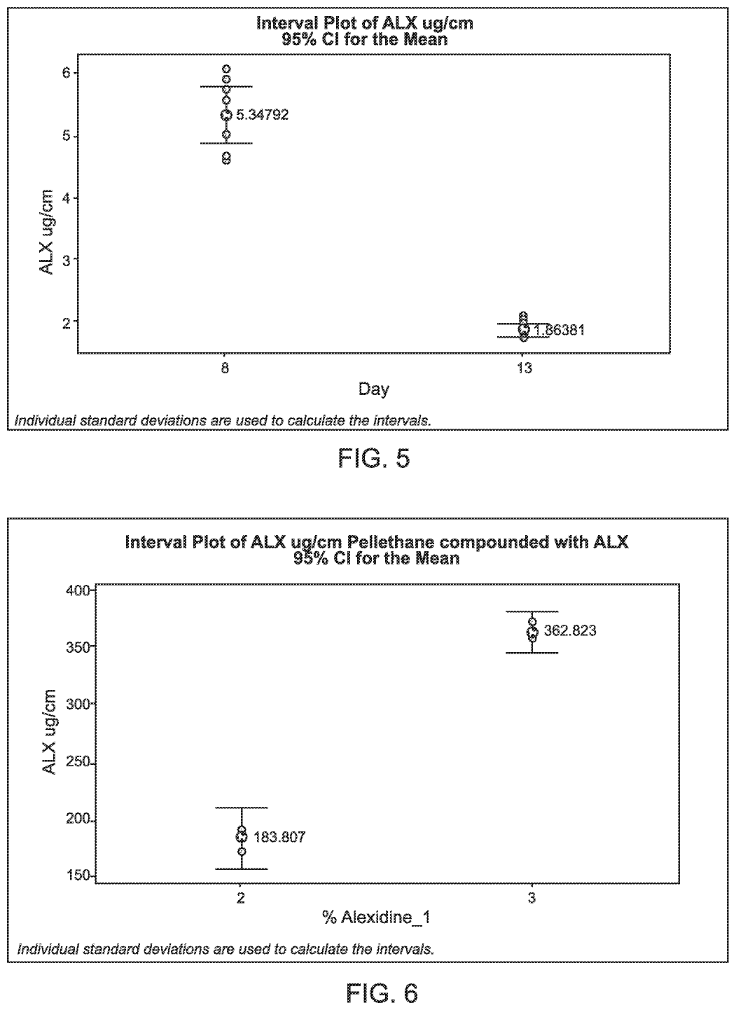

Tecoflex polyurethane material was compounded with 2.5% alexidine and 20% PEBAX followed by extruding to form 7french 3-lumen catheters, and testing for Content, Elution, and Efficacy. Content results are in FIG. 4, Elution results are in FIG. 5, and the efficacy results are in Table 2. The results in Table 2 show the catheters resulted in at least 4-Log₁₀ reduction in all of the 8 tested organisms.

TABLE 2
Antimicrobial Efficacy of the external surface of extrusions
composed of Tecoflex + 5% ALX + 20% PBAX

| | Day 14 Log₁₀ Reduction |
|---|---|
| Organism | Compared to Control |
| Candida albicans | 5.6 |
| Enterococcus faecalis | 4.3 |
| Klebsiella pneumoniae | 5.3 |
| Escherichia coli | 6.4 |
| Staphylococcus aureus | 5.1 |
| Staphylococcus epidermidis | 4.4 |
| Enterococcus cloacae | 4.0 |
| Candida tropicalis | 5.6 |

### Example 3: Pellethane+ALX (2,3%)+PBAX (0, 20%)—Formulation Composition, Content, Elution, Antimicrobial Efficacy

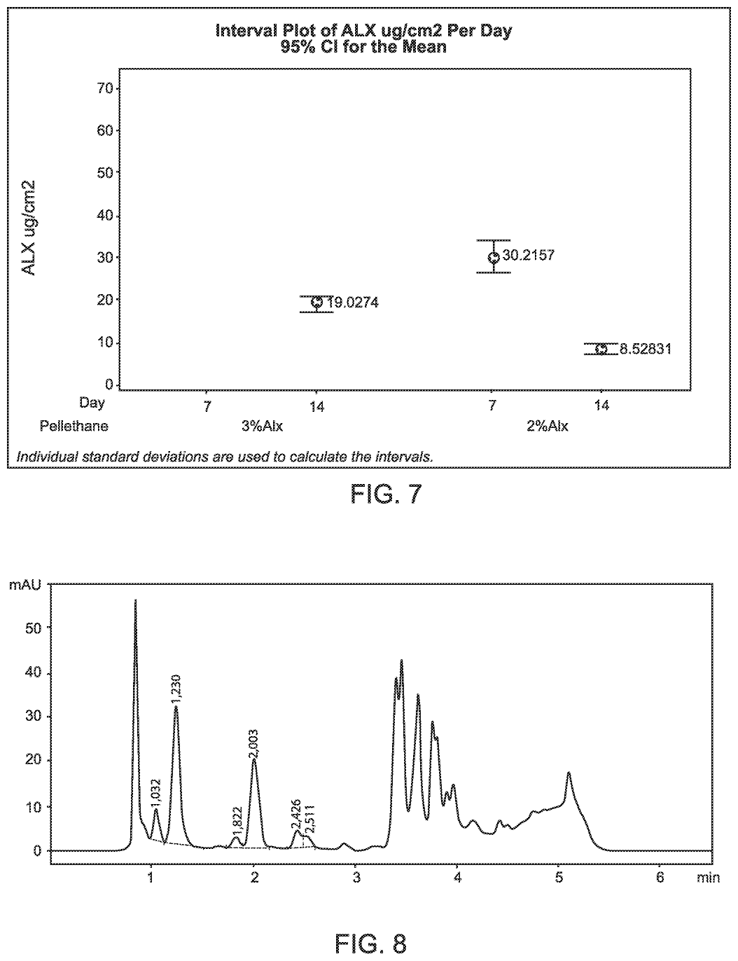

Pellethane polyurethane material was compounded with 2% or 3% alexidine, and 20% PEBAX followed by extruding to form single lumen catheter extension line extrusions, and testing for content, elution, and efficacy. The content is shown in FIG. 6, the elution is shown in FIG. 7, and the results of the efficacy are shown in Table 3. The results in Table 3 show the efficacy results to have had at least a 4 log kill on 3 out of 3 organisms.

TABLE 3
Antimicrobial Efficacy of the external surface of extrusions composed
of Pellethane + 2% or 3% ALX + 20% PBAX

Log₁₀ Reduction Compared to Control

| | 3% Alexidine + | 2% Alexidine + |
|---|---|---|
| Organism | 20% PEBAX | 20% PEBAX |
| Candida albicans | 4.5 | 4.5 |
| Enterococcus faecalis | 4.2 | 4.4 |
| Klebsiella pneumoniae | 6.4 | 6.4 |

### Example 4: Thermal Stability Assessment of CHA (Chlorhexidine Diacetate), CHD (Chlorhexidine Dihydrochloride), and ALX-D (Alexidine Dihydrochloride)

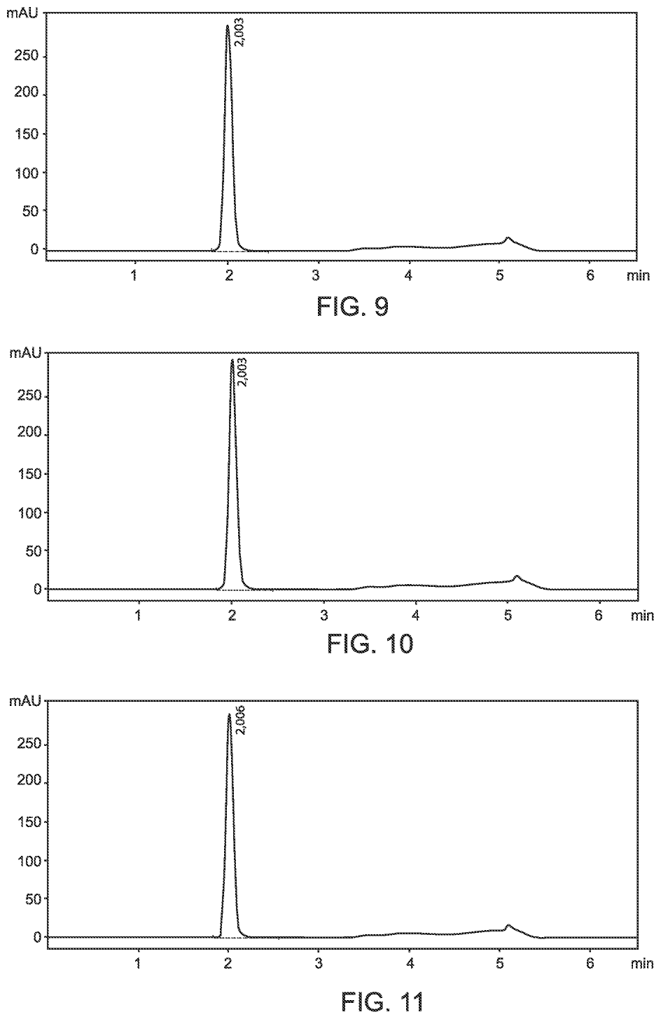

The antimicrobial agents were placed into an oven set at 210° C. for 10 mins (to mimic condition in which the antimicrobial agent would be exposed to heat during the compounding and extrusion processes). Another set of the same antimicrobial agents was not exposed to any heat. The unheated and heated samples were then examined through HPLC method for presence of degradants (extra peaks). Results of CHA and CHD are in FIGS. 8, 9, 10, and 11. CHA results in FIG. 8 is for the heated sample and there were several extra peaks from degradants detected along the baseline when compared to FIG. 9 which was the un-heated sample. FIGS. 10 and 11 which was the heated and unheated samples of CHD respectively, there was absence of any extra peaks on either sample showing thermal stability of CHD and hence the suitability for including it in the device through compounding process. Similar to CHD, ALX-D was also found stable at 210° C. for 10 mins and was found suitable for including in the device through compounding process.

### Example 5: Cooling During Compounding Process

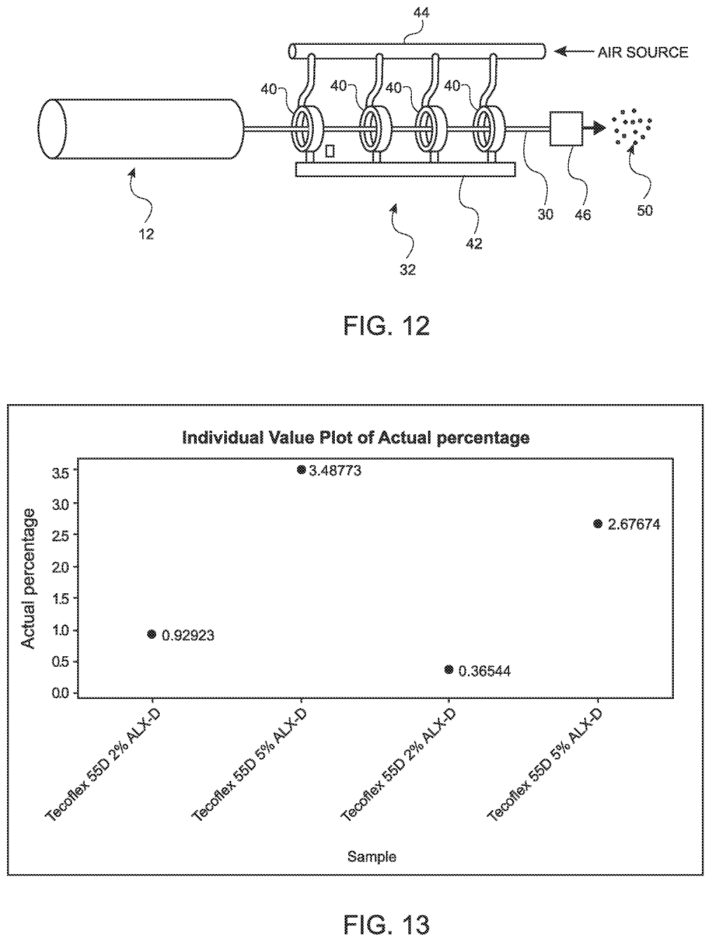

Compounding an API into a polyurethane polymer occurred at an outside vendor. The vendor uses normal compounding procedures and used an underwater pelletization set up. As shown in FIG. 12, this resulted in the loss of about 50% of the drug that was placed into the polymer matrix.

### Example 6: Air-Cooling Device

In this example, leaching of the API from the compounded polymer was reduced by eliminating the water tank and cooling the extrudate with a plurality air rings. In a first experiment, two air rings were used to cool the extrudate and the extrudate was not cool enough to cut in the pelletizer. In subsequent experiments, more air rings were added to a base and fixtures so placement of the air rings could be adjusted to a suitable distance between them. The starting % Alexidine in the experiment was 3%. FIG. 12 shows the set-up of the air rings.

As shown in FIG. 12, the compounded mixture 30 is extruded from the extruder 12. In this example, the air-cooling device 32 is a series of air rings 40 disposed on an adjustable fixture 42 and provided a supply of pressurized air via an air supply 44. Once cooled, the compounded mixture 30 is fed into a pelletizer 46. The pelletizer 46 is configured to cut and form the compounded mixture into pellets 50. The results, after implementing air-rings, reduced the Alexidine loss to 20% compared to the 50% loss observed with the water-cooling method.

### Example 7: Position of API Feeding in the Extruder

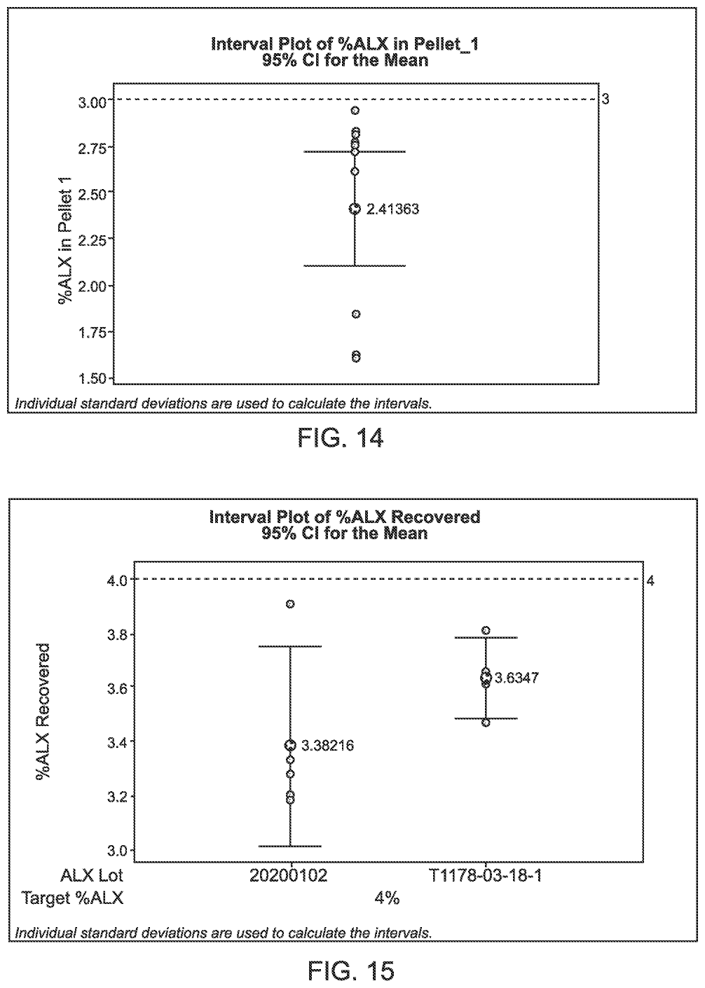

In our previous attempts at Compounding the Alexidine into the polyurethane, the base polyurethane resin, hydrophilic resin, and the Alexidine was fed into the same feed throat. This approach was leading to some of the powder getting caked onto the screw and not flowing with the resin through the compounder. To eliminate this issue, Alexidine was introduced into the melt stream of the polymer downstream of the polymer feeding throat. This indeed helped reduce the measured loss of API through the extrusion process. FIG. 14 illustrates a chart showing the measured API % is between 10 and 15% of the theoretical % in the compounding process.

### Example 8: Use of an Ionizer to Reduce API Buildup on Metal Surfaces of Extruder

Although some improvements were observed on the compounding process in reducing alexidine loss, there were still issues with alexidine attaching to the metal pieces of the feeder and feed throat, as well as seeing the Alexidine was not well distributed into the bulk of the polymer. To address this issue, an ionizer was attached to the extruder to help eliminate the static and to prevent alexidine from attaching to the metal parts. A minor increase in content was observed when the ionizer was employed. This may have been because the fan may be aerosolizing the Alexidine into the air.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A method of integrating an active pharmaceutical ingredient (API) with a thermoplastic polymer, the method comprising:
feeding the thermoplastic polymer and API into a first feed port of a multi-screw extruder;
or feeding the thermoplastic polymer into a first feed port of a multi-screw extruder;
conveying the thermoplastic polymer along the heated multi-screw extruder;
heating the thermoplastic polymer to a melt temperature of 160° C.-280° C. prior to the thermoplastic polymer being conveyed past a second feed port;
the second feed port is feeding the API into the heated screw extruder to mix with the melted thermoplastic polymer to generate a compounded mixture containing 85-100% of the starting API content;
extruding the compounded mixture from an outlet of the heated screw extruder; and
passing the extruded compounded mixture through an air-cooling device to cool the extruded compounded mixture such that the compounded mixture contains 85-100% of the starting API content.

2. The method according to claim 1, wherein the second feed port is disposed at least halfway along a length of the multi-screw extruder.

3. The method according to claim 1, wherein the air-cooling device provides a flow of air at a flow rate of 2-20 meters per second.

4. The method according to claim 1, further including:
conveying the compounded mixture from the outlet with a conveyer belt; and
cooling the compounded mixture by cooling at least one of the conveyer belt and air contacting the compounded mixture with a chilled platen.

5. The method according to claim 1, wherein the cooled compounded mixture is pelletized resulting in pellets containing 85-100% of the starting API content.

6. The method according to claim 1, wherein the API is an antimicrobial, antithrombogenic and/or anti-inflammatory drug which is thermally stable at a temperature range of 200° C.-280° C.

7. The method according to claim 6, wherein the API is a salt of chlorhexidine or a salt of alexidine.

8. The method according to claim 1, wherein the thermoplastic polymer includes a hydrophilic polyurethane polymer with 5-40% water uptake.

9. A medical device comprising:
a thermoplastic polymer integrated with an active pharmaceutical ingredient (API), wherein a method of integrating the API with the thermoplastic polymer comprises:
feeding the thermoplastic polymer and API into a first feed port of a twin-screw extruder;
or feeding the thermoplastic polymer into a first feed port of a twin-screw extruder, conveying the thermoplastic polymer along the heated multi-screw extruder, heating the thermoplastic polymer to a melt temperature of 160° C.-280° C. prior to the thermoplastic polymer being conveyed past a second feed port;
the second feed port is feeding the API into the heated multi-screw extruder to mix with the melted thermoplastic polymer to generate a compounded mixture containing 85-100% of the starting API content;
extruding the compounded mixture from an outlet of the heated screw extruder; and
passing the extruded compounded mixture through an air-cooling device to cool the extruded compounded mixture such that the compounded mixture contains 85-100% of the starting API content.

10. The medical device according to claim 9, wherein the second feed port is disposed at least halfway along a length of the multi-screw extruder.

11. The medical device according to claim 9, wherein the air-cooling device provides a flow of air at a flow rate of 2-20 meters per second.

12. The medical device according to claim 9, further including:
a conveyer belt configured to convey the compounded mixture from the outlet; and
a chilled platen configured to facilitate cooling the compounded mixture by cooling at least one of the conveyer belt and air contacting the compounded mixture.

13. The medical device according to claim 9, wherein the cooled compounded mixture is pelletized resulting in pellets containing 85-100% of the starting API content.

14. The medical device according to claim 9, wherein the API is an antimicrobial, antithrombogenic and/or anti-inflammatory drug which is thermally stable at a temperature range of 200° C.-280° C.

15. The medical device according to claim 14, wherein the API is a salt of chlorhexidine or a salt of alexidine.

16. The medical device according to claim 9, wherein the thermoplastic polymer includes a hydrophilic polyurethane polymer with 5-40% water uptake.

17. The medical device according to claim 9, further comprising a second thermoplastic polymer without API, wherein the compounded thermoplastic polymer with a bulk distributed API is co-extruded with the second polymer without API.

18. The medical device according to claim 17, wherein the compounded thermoplastic polymer with bulk distributed API is extruded on an inside portion of the medical device and the second thermoplastic polymer without API is extruded on an outside portion of the medical device.

19. The medical device according to claim 17, wherein the compounded thermoplastic polymer with bulk distributed API is extruded along a first longitudinal portion of the medical device and the second thermoplastic polymer without API is extruded along a second longitudinal portion of the medical device, the second thermoplastic polymer without API being configured to be transparent to provide a viewing port for a user to see within the medical device.
